(19) United States
(12) Patent Application Publication
(10) Pub. No.: US 2008/0287666 A1
(43) Pub. Date: Nov. 20, 2008

(54) DETECTING NUCLEIC ACID STRANDS AND INTER-SUBSTANCE BINDING OR INTERACTION DETECTING METHOD

(75) Inventors: Yuuki Watanabe, Kanagawa (JP); Masatsugu Ueno, Tokyo (JP)

Correspondence Address: BELL, BOYD & LLOYD, LLP, P. O. BOX 1135, CHICAGO, IL, 60690, US

(73) Assignee: SONY CORPORATION, Tokyo (JP)

(21) Appl. No.: 12/120,119

(22) Filed: May 13, 2008

(30) Foreign Application Priority Data
May 14, 2007 (JP) 2007-128188

(51) Int. Cl. C07H 21/04

(52) U.S. Cl. 536/24.3

(57) ABSTRACT

Disclosed herein is a detecting nucleic acid strand including: a first nucleic acid strand having a first base sequence region capable of functioning as an aptamer, and a second nucleic acid strand having a second base sequence region, which is complementary to the first base sequence region and forms a complementary strand with the first base sequence region, wherein the detecting nucleic acid strand is designed such that, when a predetermined substance interacts with the first base sequence region, the complementary strand of the first and second base sequence regions dissociates into single strands.

### CROSS REFERENCES TO RELATED APPLICATIONS

The present application claims priority to Japanese Patent Application JP 2007-128188 filed in the Japan Patent Office on May 14, 2007, the entire contents of which being incorporated herein by reference.

### BACKGROUND

The present application relates to detecting nucleic acid strands and detection method usable for the detection of specific substances or bindings or interactions between substances. More specifically, the present application is concerned with detecting nucleic acid strands capable of detecting specific substances or bindings or interactions between substances and also with a detection method for bindings or interactions between substances.

Technologies that detect proteins, organic small molecules, nucleic acids, multimeric complexes, cells, cell tissues, metal ions, microorganisms, viruses and the like bear an extremely important part in research, developments and the like in a wide variety of fields. Developments of detection technologies are now under way in all areas such as, for example, the diagnosis of specific diseases, drug developments, hygienic control of foods and the like, and cleanup and restoration of environmental pollutions.

Among these, advanced especially in recent years are technologies that allow an interaction or reaction to proceed between substance on a surface (interface) of a substrate or the like and detect it by a physical means, optical means or the like. These technologies have already found increasing utility as critical technologies in areas such as the diagnosis of diseases, screening of compounds such as drugs, forensic medicine, comprehensive analysis of genetic information, function analysis of biosubstances, proteome analysis, and analysis of in vivo interactions.

As detection technologies, JP-A-08-333398 discloses an immunoassay method making use of a polyclonal or monoclonal antibody which specifically recognizes cortisol (a kind of hormone) in urine; JP-A-2007-000009 discloses a method for easily and accurately detecting target nucleic acid molecules by using a degradative enzyme specific to double-stranded nucleic acids; JP-A-2006-133098 discloses a detection method of a protein, which detects the existence or non-existence of the protein by inserting an active electrode in a separation medium, through which a protein sample has been subjected to electrophoresis, to oxidize functional groups of the protein and measuring the resulting electric current; JP-A-2002-296274 discloses a method for detecting tumor cells and their precursors in an uterocervical smear preparation by concurrently staining and detecting at least two different molecular markers, which indicate disease-associated variations in gene expression, with an antibody or nucleic acid probe; and JP-A-2006-262775 discloses a detection method of microorganism cells, which is characterized by bringing a luminescence sensitizer into contact with a sample of microorganism cells to increase the intensity of fluorescence to be emitted by the microorganism cells.

Here, a description is made about “aptamer” which is relevant to the present application. The term “aptamer” means a nucleic acid molecule or peptide having a function that allows the nucleic acid molecule or peptide to specifically bind to a specific substance such as a protein, an organic small molecule, a nucleic acid, a multimeric complex, cells, a cell tissue, metal ions, a microorganism, a virus or the like. An “aptamer” can bind to any object without limitation, and is allowed to bind to an object with high affinity and specificity. Its mass synthesis is easy, and its acting mechanism is simple. It can, therefore, be used widely in areas such as structural proteomics, target analysis, target validation, and drug developments.

For the above-described reasons, technological developments on various aptamers are under way in recent years. For example, JP-A-2006-320289 discloses an RNA aptamer, which binds specifically to fibrils derived from abnormal prion protein (mSAF) and is usable for the diagnosis, treatment or prevention of prion disease; and JP-A-2007-082543 discloses an RNA aptamer which inhibits Escherichia coli release factors.

### SUMMARY

The above-described substance detection methods all have both merits and demerits. For example, each of them involves one or more problems in that it is not suited for the detection of small molecules, it cannot detect if a sample is in a trace amount, and/or it requires a special step to avoid deactivation of a substance. Accordingly, further developments are still desired.

An aspect of the present application is, therefore, to provide a novel detecting nucleic acid strand which in turn can provide a substance detection method not known to date.

The present inventors conducted extensive research with a view to developing a substance detection method making use of the property of an aptamer. As a result, the present inventors radically changed the idea of measuring a change in a probe or target itself that had been practiced in the conventional methods, and have found a novel method capable of detecting a target by contriving to make a substance, which is different from an aptamer or the target, change upon binding of the aptamer with the target and measuring that change.

In an embodiment, there is thus provided a detecting nucleic acid strand comprising: a first nucleic acid strand having a first base sequence region capable of functioning as an aptamer, and a second nucleic acid strand having a second base sequence region, which is complementary to the first base sequence region and forms a complementary strand with the first base sequence region, wherein the detecting nucleic acid strand is designed such that, when a predetermined substance interacts with the first base sequence region, the complementary strand of the first and second base sequence regions dissociates into single strands.

No particular limitation is imposed on a method for detecting the dissociation of the complementary strand of the first and second base sequence regions into single strands. As an example, however, it is possible to design such that the detecting nucleic acid strand comprises a probe.

No particular limitation is imposed on the position, function or the like of the probe, insofar as it has a function to provide a detection signal indicating the dissociation of the complementary strand of the first and second base sequence regions into single strands. For example, the probe can be a material, which is held on the second nucleic acid strand of the detecting nucleic acid strand and is capable of producing a detection signal. Specific examples can include a dielectric and a fluorescent material.

The probe may also be formed of two or more materials. Illustrative can be a probe comprising a fluorescent material labeled on the second nucleic acid strand and a quencher labeled on the first nucleic acid stand and capable of quenching the fluorescent material while the complementary strand of the first and second base sequence regions exists.

In an embodiment, there is also provided a method for detecting an interaction between a first base sequence region capable of functioning as an aptamer and a predetermined substance by using at least the above-described detecting nucleic acid strand.

In the above-described method, the first nucleic acid strand can be immobilized at an end thereof on a solid-phase surface.

No particular limitation is imposed on a detection method of the binding or interaction between the first base sequence region and the predetermined substance. For example, however, the binding or interaction between the first base sequence region and the predetermined substance can be detected by measuring a change in a dielectric constant available from a dielectric held on the second nucleic acid strand and detecting the dissociation of the complementary strand into the single strands.

The binding or interaction between the first base sequence region and the predetermined substance can also be detected by measuring a change in a weight of the detecting nucleic acid strand and detecting the dissociation of the complementary strand into the single strands.

Further, the binding or interaction between the first base sequence region and the predetermined substance can also be detected by measuring a reduction in an intensity of fluorescence from an intercalator bound or adsorbed on the complementary strand of the first and second base sequence regions in the detecting nucleic acid strand and capable of emitting fluorescence and detecting the dissociation of the complementary strand into the single strands.

A description will now be made about certain technical terms relating to the present application. The term “aptamer” as used herein means a nucleic acid molecule or peptide having a function that allows the nucleic acid molecule or peptide to specifically bind to a specific substance such as a protein, an organic small molecule, a nucleic acid, a multimeric complex, cells, a cell tissue, metal ions, a microorganism, a virus or the like.

The term “probe” as used herein embraces all materials each of which can acquire a detection signal for the detection of a predetermined substance. Examples can include dielectrics, beads having desired weights, fluorescent materials, radioactive materials, intercalators, and the like.

The term “quencher” as used herein means a material, which is an excitation energy absorber and has a function to inhibit the emission of fluorescence from a nearby fluorescent material.

The term “intercalator” as used herein means a material that binds to a complementary strand site in a double-stranded nucleic acid to emit fluorescence or the like. Examples can include “POPO-1” (trade name, product of Molecular Probes, Inc.), “TOTO-3” (trade name, product of Invitrogen Corporation), “SYBR™ GREEN I” (product of Invitrogen Corporation), “PICOGREEN™”, (product of Molecular Probes, Inc.), and Hoechst 33258.

The use of the detecting nucleic acid strand according to the present application makes it possible to perform simple and accurate detection of a predetermined substance irrespective of its size, amount, kind and the like. Further, the detecting nucleic acid strand according to the present application can minimize a reduction in the activity of the predetermined substance, and therefore, does not require to conduct any additional step for the inhibition of the reduction in the activity. Furthermore, the detecting nucleic acid strand according to an embodiment can detect a binding or interaction between substances with high accuracy.

The method according to an embodiment to detect an interaction between substances can be applied to the analysis of a function of an aptamer, the analysis of a function of a predetermined substance after its binding to the aptamer or the screening of a substance, and is expected to make contributions to all areas such as the diagnosis of diseases, screening of compounds such as drugs, forensic medicine, comprehensive analysis of genetic information, function analysis of biosubstances, proteome analysis, analysis of in vivo interactions, hygienic control of foods and the like, and cleanup and restoration of environmental pollutions.

Additional features and advantages are described herein, and will be apparent from the following Detailed Description and the figures.

### BRIEF DESCRIPTION OF THE FIGURES

FIGS. 1A and 1B are schematic illustrations of a detecting nucleic acid strand according to a first embodiment of the first aspect of the present application and a method according a first embodiment of the second aspect of the present application for detecting a substance with the detecting nucleic acid strand;

FIGS. 2A to 2C are schematic illustrations of a detecting nucleic acid strand according to a second embodiment of the first aspect of the present application and a method according a second embodiment of the second aspect of the present application for detecting a substance with the detecting nucleic acid strand;

FIGS. 3A and 3B are schematic illustrations of a detecting nucleic acid strand according to a third embodiment of the first aspect of the present application and a method according to a third embodiment of the second aspect of the present application for detecting a substance with the detecting nucleic acid strand;

FIGS. 4A and 4B are schematic illustrations of a detecting nucleic acid strand according to a fourth embodiment of the first aspect of the present application and a method according to a fourth embodiment of the second aspect of the present application for detecting a substance with the detecting nucleic acid strand;

FIGS. 5A and 5B are schematic illustrations of a method according to a fifth embodiment of the second aspect of the present application for detecting an interaction between predetermined substances;

FIGS. 6A and 6B are schematic illustrations of a method according to a sixth embodiment of the second aspect of the present application for detecting an interaction between predetermined substances; and

FIGS. 7A to 7C are schematic illustrations of a method according to a seventh embodiment of the second aspect of the present application for the detection of a binding or interaction between substances as applied to the analysis of a function of an aptamer, the analysis of a function of a predetermined substance after its binding to the aptamer or the screening of a substance.

### DETAILED DESCRIPTION

With reference to the accompanying drawings, a description will hereinafter be made about preferred embodiments of the present application. It is, however, to be noted that the embodiments to be described hereinafter merely illustrate representative embodiments of the present application by way of example and that the scope of the present application shall not be narrowly interpreted by the following examples.

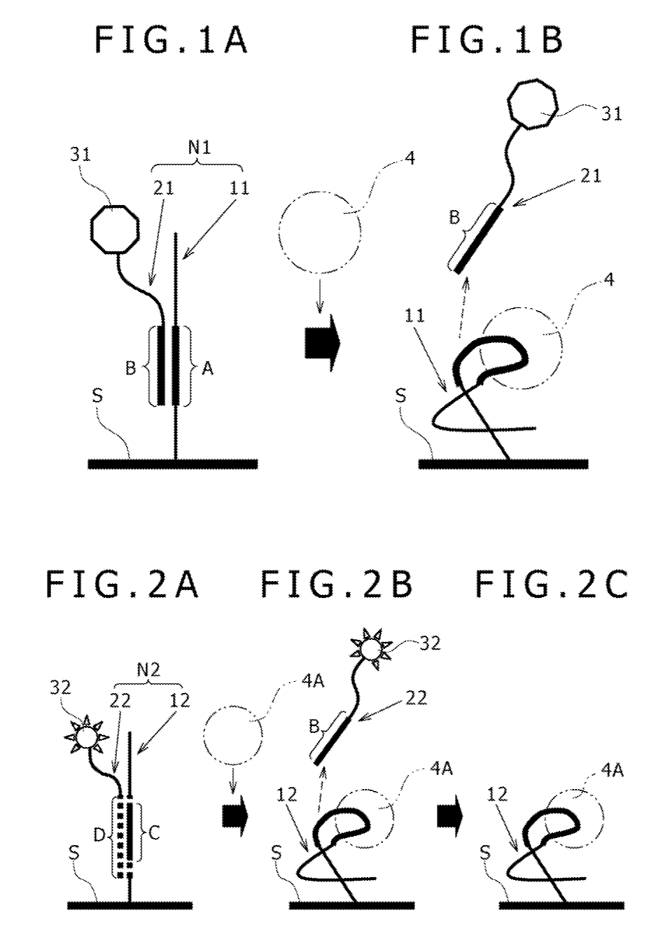

Referring firstly to FIGS. 1A and 1B, a description will be made of a detecting nucleic acid strand N1 according to the first embodiment of the first aspect of the present application and a method according the first embodiment of the second aspect of the present application for detecting a predetermined substance 4 with the detecting nucleic acid strand N1.

Described roughly, the detecting nucleic acid strand N1 according to the first embodiment of the first aspect of the present application is provided at least with a first nucleic acid strand 11 and a second nucleic acid strand 21.

The first nucleic acid strand 11 has, as the first base sequence region, a base sequence region A which functions as an aptamer. In FIGS. 1A and 1B, the first nucleic acid strand 11 is immobilized at an end thereof on a solid-phase surface S (such as beads or the like; this applied equally hereinafter) although it is not limited to such an immobilized form. As will be described subsequently with reference to FIGS. 4A and 4B, it may be in a free form. Upon immobilizing the first nucleic acid strand 11 at the end thereof on the solid-phase surface S, no particular limitation is imposed on a method for its immobilization, and known methods are all usable. Illustrative are avidin-biotin binding and coupling reactions (for example, diazo-coupling reaction).

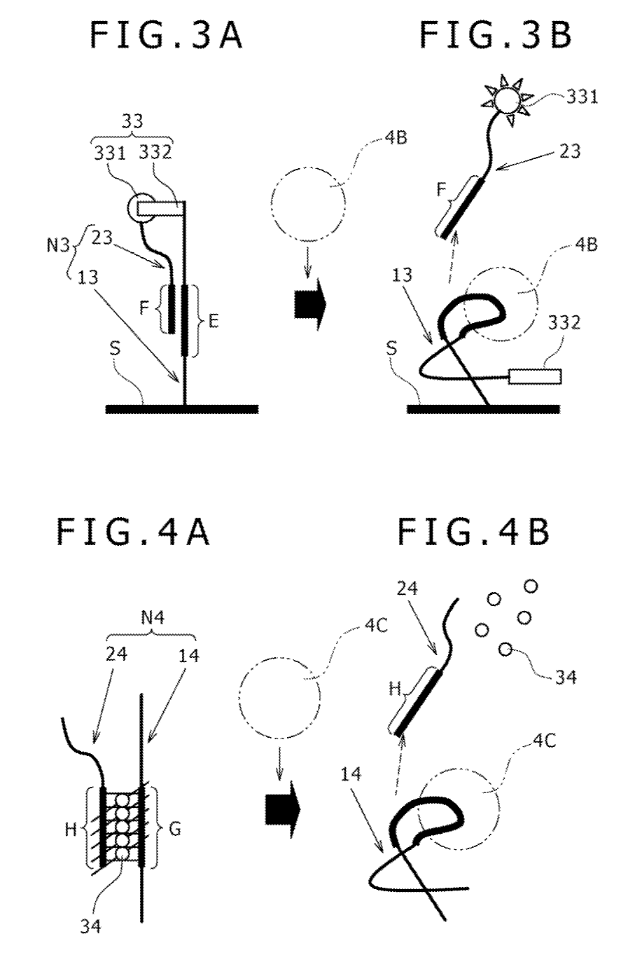

The second nucleic acid strand 21 has, as the second base sequence region, a base sequence region B complementary to the base sequence region A. In FIGS. 1A and 1B, a base sequence region complementary to the whole base sequence region A is shown as an example of the base sequence region B. The base sequence region B is, however, not limited to such a base sequence region, and as shown in FIGS. 2A to 2C, the base sequence region B may be complementary to a base sequence region longer than the base sequence region A. Further, the base sequence region B may be complementary to at least a part of the base sequence region A as depicted in FIGS. 3A and 3B.

In FIGS. 1A and 1B, the second nucleic acid strand 21 is modified at an opposite end thereof with a probe, that is, a dielectric 31. It is, however, unnecessary to modify the second nucleic acid strand 21 with the probe beforehand. For example, a modification, labeling or the like may be applied shortly before a detection. No particular limitation is imposed on the kind of the probe, although a substance capable of acquiring a physical or chemical detection signal is preferred. In addition to the dielectric 31, all known probes such as beads having known weights, fluorescent materials, radioactive materials and intercalators can be used.

It is the detecting nucleic acid strand N1 according to the first embodiment of the first aspect of the present application that the above-described first nucleic acid strand 11 and second nucleic acid strand 21 are in a double-stranded form.

In FIGS. 1A and 1B, numeral 4 designates a predetermined substance which specifically binds to the base sequence region A. No particular limitation is imposed on the predetermined substance 4 to be detectable, insofar as it is a substance that specifically binds or interacts to the base sequence region A which functions as an aptamer. Examples can include proteins, organic small molecules, nucleic acids, multimeric complexes, cells, cell tissues, metal ions, microorganisms, viruses, and the like.

In a place of reaction or interaction, the detecting nucleic acid strand N1 is ready for reaction or interaction (see FIG. 1A). When the predetermined substance 4 is charged there, the base sequence region A and the predetermined substance 4 bind to each other to result in dissociation of the second nucleic acid strand 21 as illustrated in FIG. 1B when the associativity between the predetermined substance 4 and the base sequence region A is more dominant over the associativity between the predetermined substance 4 and the base sequence region B. The strength of associativity can be adjusted by modifying the length of the base sequence region A or B and/or the GC content.

As the second nucleic acid strand 21 carries the dielectric 31 thereon for its modification, the dielectric constant on the solid-phase surface significantly changes when the second nucleic acid strand 21 dissociates. The predetermined substance 4 can, therefore, be detected by measuring a change in dielectric constant with a surface plasmon resonance sensor (SPR sensor) or the like.

When the probe carried on the second nucleic acid strand 21 for its modification is a bead having a desired weight, the predetermined substance 4 can also be detected by measuring a change in weight in accordance with the quartz crystal microbalance method (QCM method) or the like.

Different from commonly-employed probe nucleic acids, the detecting nucleic acid strand N1 according to the first embodiment of the first aspect of the present application is designed such that only the base sequence region A in the first nucleic acid strand 11 forming the double strand binds or interacts to the predetermined substance 4 and the other nucleic acid strand forming the double strand, i.e., the second nucleic acid strand 21 dissociates at the same time. It is, therefore, unnecessary to perform labeling or the like on the predetermined substance 4. Further, the detection of the predetermined substance 4 is performed by measuring a change in the dissociating second nucleic acid strand 21 rather than measuring a change in the predetermined substance 4 itself. It is, accordingly, possible to detect the predetermined substance 4 with high sensitivity even when the predetermined substance 4 is a small molecule or is in a trace amount.

With reference to FIGS. 2A to 2C, a description will next be made of a detecting nucleic acid strand N2 according to the second embodiment of the first aspect of the present application and a method according the second embodiment of the second aspect of the present application for detecting a predetermined substance 4A with the detecting nucleic acid strand N2.

The detecting nucleic acid strand N2 according to the second embodiment of the first aspect of the present application is in a form that a base sequence region D as the second base sequence region in a second nucleic acid strain 22 complimentarily forms a double strand with a base sequence region which as the first base sequence region, is longer than the base sequence region C in a first nucleic acid strain 12. Further, a fluorescent material 32 as an illustrative probe is labeled to one end of the second nucleic acid strand 22. No particular limitation is imposed on the kind of the fluorescent material 32. For example, however, any known fluorescent material such as a florescent dye, e.g., Cy3 or Cy5 or a fluorescent protein, e.g., luciferase or GFP can be used.

Similar to the first embodiments shown in FIGS. 1A and 1B, the detecting nucleic acid strand N2 is ready for reaction or interaction in a place of reaction or interaction (see FIG. 2A). When a predetermined substance 4A is charged there, the base sequence region C and the predetermined substance 4A bind to each other to result in dissociation of the second nucleic acid strand 22 as illustrated in FIG. 2B.

The predetermined substance 4A can be detected by washing the place of reaction or interaction and then determining whether or not fluorescence is emitted by fluorescence excitation light of a predetermined wavelength. Described specifically, when the predetermined substance 4A which binds to or interacts with the base sequence region C exists, the second nucleic acid strands dissociates so that as illustrated in FIG. 2C, the second nucleic acid strand 22 is eliminated by subsequent washing and no fluorescence is emitted even when fluorescence excitation light of the predetermined wavelength is irradiated. When the predetermine substance 4A which binds to or interacts with the base sequence region C does not exist, the second nucleic acid strand 22 does not dissociate and the detecting nucleic acid strand N2 remains in the form of the double strand. As the detecting nucleic acid strand N2 is not eliminated by washing, fluorescence is emitted when fluorescence excitation light of the predetermined wavelength is irradiated.

Referring next to FIGS. 3A and 3B, a description will hereinafter be made of a detecting nucleic acid strand N3 according to the third embodiment of the first aspect of the present application and a method according the third embodiment of the second aspect of the present application for detecting a predetermined substance 4B with the detecting nucleic acid strand N2.

The detecting nucleic acid strand N3 according to the third embodiment of the first aspect of the present application is in a form that a base sequence region F as the second base sequence region in a second nucleic acid strain 23 complimentarily forms a double strand with a part of a base sequence region E as the first base sequence region in a first nucleic acid strain 13. Employed as a probe is one composed of a fluorescent material 331 and a quencher 332 capable of quenching the florescent material 331. It is designed that the fluorescent material 331 is labeled to one end of the second nucleic acid strand 23 and the quencher 332 is labeled to one end of the first nucleic acid strand 13 to quench the fluorescent material 331 as long as the double strand is formed.

Similar to the embodiments shown in FIGS. 1A and 1B and FIGS. 2A to 2C, the detecting nucleic acid strand N3 is ready for reaction or interaction in a place of reaction or interaction with the fluorescent material 331 being in a quenched state (see FIG. 3A). When a predetermined substance 4B is charged there, a base sequence region E and the predetermined substance 4B bind to each other to dissociate a second nucleic acid strand 23 as illustrated in FIG. 3B.

Upon dissociation of the first nucleic acid strand 13 and the second nucleic acid strand 23 from each other, the fluorescent material 331 and the quencher 332 separate from each other so that the fluorescent material 331 becomes ready to emit fluorescence. Measurement of an emission of fluorescence by fluorescent excitation light of a predetermined wavelength, therefore, makes it possible to detect the predetermined substance 4B.

The use of a quencher as in the third embodiments can bring about a merit that a washing step such as that conducted in the second embodiments shown in FIGS. 2A to 2C is no longer required.

With reference to FIGS. 4A and 4B, a description will hereinafter be made of a detecting nucleic acid strand N4 according to the fourth embodiment of the first aspect of the present application and a method according the fourth embodiment of the second aspect of the present application for detecting a predetermined substance 4C with the detecting nucleic acid strand N4.

The detecting nucleic acid strand N4 according to the fourth embodiment is not immobilized on any solid-phase surface, but is in a free state. As an illustrative probe, an intercalator 34 is used and is bound or adsorbed on a complementary strand of a base sequence region G as the first base sequence region in a first nucleic acid strand 14 and a base sequence region H as the second base sequence region in a second nucleic acid strand 24.

Similar to the embodiments shown in FIGS. 1A and 1B, FIGS. 2A to 2C and FIGS. 3A and 3B, the detecting nucleic acid strand N4 is ready for reaction or interaction in a place of reaction or interaction (see FIG. 4A). When a predetermined substance 4C is charged there, the base sequence region G and the predetermined substance 4C bind to each other to result in dissociation of the second nucleic acid strand 24 as illustrated in FIG. 4B.

Upon dissociation of the first nucleic acid strand 14 and the second nucleic acid strand 24 from each other, the intercalator 34 also dissociates. Measurement of a reduction in fluorescence emission under irradiation of fluorescence excitation light or the like of a predetermined wavelength, therefore, makes it possible to detect the predetermined substance 4C.

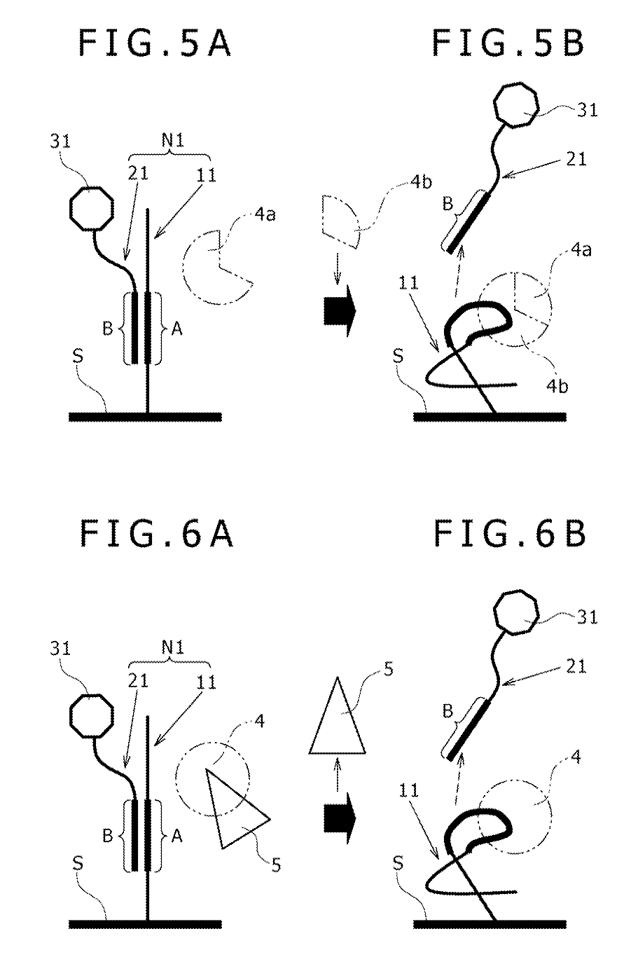

FIGS. 5A and 5B illustrate a method according to a fifth embodiment of the second aspect of the present application for detecting an interaction between predetermined substances 4a,4b. In this fifth embodiment, a description will be made using, as a detecting nucleic acid strand, the detecting nucleic acid strand N1 shown in FIGS. 1A and 1B. The firth embodiment is, however, not limited to the use of the detecting nucleic acid strand N1. For example, any one of the above-described detecting nucleic acid strands according to the first to fourth embodiments can also be used as desired.

The predetermined substance indicated at sign 4a in FIGS. 5A and 5B cannot bind to the base sequence region A as long as it exists as is. When it interacts with the other predetermined substance 4b, however, its properties are changed such that it can bind to the base sequence region A.

In a place of reaction or interaction, the detecting nucleic acid strand N1 and the predetermined substance 4a are ready for reaction or interaction (see FIG. 5A). When the predetermined substance 4b is charged there, an interaction takes place between the predetermined substance 4a and the predetermined substance 4b. The thus-interacted predetermined substance 4a,4b then binds to the base sequence region A to result in dissociation of the second nucleic acid strand 21.

As the second nucleic acid strand 21 carries the dielectric 31 thereon for its modification, the dielectric constant on the solid-phase surface significantly changes when the second nucleic acid strand 21 dissociates. The interaction between the predetermined substance 4a and the predetermined substance 4b can, therefore, be detected by measuring a change in dielectric constant, for example, with a surface plasmon resonance sensor (SPR sensor) or the like.

FIGS. 6A and 6B are schematic illustrations of a method according to a sixth embodiment of the second aspect of the present application for detecting an interaction between the predetermined substance 4 and a binding inhibition substance 5. In this sixth embodiment, a description will also be made using, as a detecting nucleic acid strand, the detecting nucleic acid strand N1 shown in FIGS. 1A and 1B. The sixth embodiment is, however, not limited to the use of the detecting nucleic acid strand N1. For example, any one of the above-described detecting nucleic acid strands according to the first to fourth embodiments can also be used as desired.

In FIG. 6A, the binding inhibition substance 5 inhibits binding between the predetermined substance 4 and the base sequence region A. When the binding inhibition substance 5 dissociates from the predetermined substance 4 or the function of the binding inhibition substance 5 is lost under certain action, the predetermined substance 4 binds to the base sequence region A to result in dissociation of the second nucleic acid strand 21.

As the second nucleic acid strand 21 carries the dielectric 31 thereon for its modification, the dielectric constant on the solid-phase surface significantly changes when the second nucleic acid strand 21 dissociates. Dissociation of the binding inhibition substance 5 from the predetermined substance 4 or a functional failure of the binding inhibition substance 5 can, therefore, be detected by measuring a change in dielectric constant, for example, with a surface plasmon resonance sensor (SPR sensor) or the like.

The method according to the sixth embodiment can be applied to the screening of a function inhibitor such as the binding inhibition substance 5. When the predetermined substance 4 exists in a state that it is inhibited from binding to the detecting nucleic acid strand N1, specifically the base sequence region A by the binding inhibition substance 5 as shown in FIG. 6A and an unillustrated substance capable of acting as a function inhibitor for the binding inhibition substance 5 is then fed to inhibit the function of the binding inhibition substance 5, the predetermined substance binds to the base sequence region A. This binding results in dissociation of the second nucleic acid strand 21 so that the dielectric constant on the solid-phase surface significantly changes. As readily appreciated from the foregoing, the detecting nucleic acid strand N1 according to the first embodiment of the first aspect of the present application can be used in the screening of a function inhibitor for the binding inhibition substance 5.

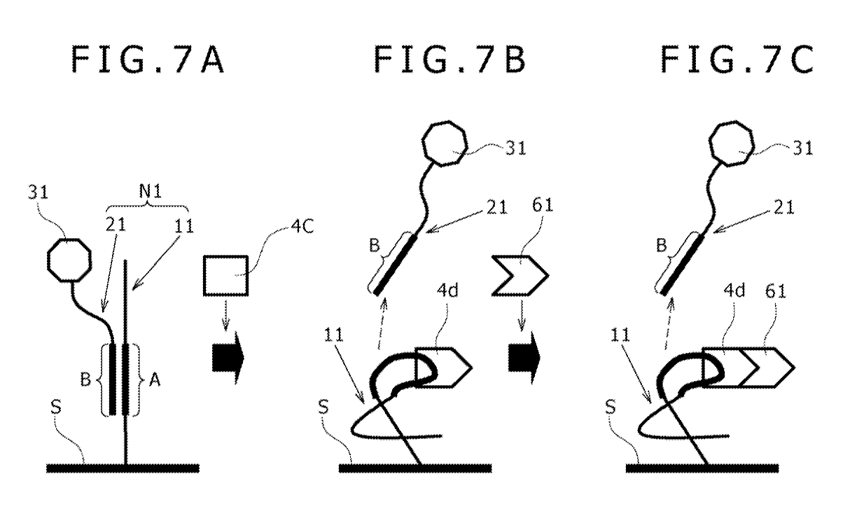

The above-described methods making use of the respective detecting nucleic acid strands can also be applied to the analysis of functions and the like to be described subsequently herein. One example of such applications will hereinafter be described with reference to FIGS. 7A to 7C.

FIGS. 7A to 7C illustrates a method according to a seventh embodiment of the second aspect of the present application for the detection of a binding or interaction between substances as applied to the analysis of a function of an aptamer, the analysis of a function of a predetermined substance after its binding to the aptamer or the screening of a substance. In this seventh embodiment, a description will also be made using, as a detecting nucleic acid strand, the detecting nucleic acid strand N1 shown in FIGS. 1A and 1B. The seventh embodiment is, however, not limited to the use of the detecting nucleic acid strand N1. For example, any one of the above-described detecting nucleic acid strands according to the first to fourth embodiments can also be used as desired.

As mentioned above, the detecting nucleic acid strand N1 is ready for reaction or interaction in a place of reaction or interaction (see FIG. 7A). When a predetermined substance 4c is charged there, the predetermined substance 4c and the base sequence region A bind to each other to result in dissociation of the second nucleic acid strand 21 as illustrated in FIG. 7B. The binding of the predetermined substance 4c to the base sequence region A, which functions as an aptamer, can firstly be confirmed by measuring a change in dielectric constant or the like at this time.

An aptamer is equipped with a function that its binds to various substances to affect their effects. When the predetermined substance 4c which normally does not undergo any interaction, for example, with a substance 61 becomes capable of interacting with the substance 61 subsequent to its binding to the aptamer (base sequence region A), this aptamer (base s sequence region A) is then appreciated to have a function to modify the predetermined substance 4c into a substance 4d which can interact with the substance 61.

When it is unknown what function the predetermined substance 4c would be provided with subsequent to its binding to the aptamer, the method according to the seventh embodiment makes it possible to perform a functional analysis of the substance 4d obtained by the binding of the predetermined substance 4c to the aptamer (base sequence region A). If the predetermined substance c can be ascertained to have become capable of interacting with the substance 61 subsequent to its binding to the aptamer (base sequence region A) as illustrated in FIG. 7C, the substance 4d resulted from the binding of the predetermined substance 4c to the aptamer (base sequence region A) is appreciated to be equipped with such properties as permitting its interaction with the substance 61.

Further, the use of the principle of the above-described method also makes it possible to perform screening of the substance 61 available from the interaction of the predetermined substance 4c with the aptamer (base sequence region A).

In the above-described applications, the existence or non-existence of the interaction between the substance 61 and the substance 4c can be determined by a conventionally-known method. As the weight changes through the interaction between the substance 61 and the substance 4d, the interaction can be detected by the quartz crystal microbalance method (QCM method) or the like. The interaction can also be detected by a method that a labeling material, such as a fluorescent material, radioactive material or intercalator, is bound to or adsorbed on the substance 61 or substance 4d or by a method that as in the principle of PRET, the color or the like of fluorescence from the substance 61 or substance 4d itself is caused to change by the interaction between the substances.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A detecting nucleic acid strand comprising:
a first nucleic acid strand having a first base sequence region capable of functioning as an aptamer, and
a second nucleic acid strand having a second base sequence region, which is complementary to said first base sequence region and forms a complementary strand with said first base sequence region,
wherein said detecting nucleic acid strand is designed such that, when a predetermined substance interacts with said first base sequence region, said complementary strand of said first and second base sequence regions dissociates into single strands.

2. The detecting nucleic acid strand according to claim 1, further comprising a probe usable to detect the dissociation of said complementary strand of said first and second complementary base sequence regions into said single stands.

3. The detecting nucleic acid strand according to claim 2, wherein said probe is a material, which is held on said second nucleic acid strand and can produce a detection signal.

4. The detecting nucleic acid strand according to claim 3, wherein said material is a dielectric.

5. The detecting nucleic acid strand according to claim 3, wherein said material is a fluorescent material.

6. The detecting nucleic acid strand according to claim 2, wherein said probe comprises:
a fluorescent material labeled on said second nucleic acid strand, and
a quencher labeled on said first nucleic acid stand and capable of quenching said fluorescent material while said complementary strand of said first and second base sequence regions exists.

7. A method for detecting an interaction between a first base sequence region capable of functioning as an aptamer and a predetermined substance by using at least a detecting nucleic acid strand according to claim 1.

8. The method according to claim 7, wherein said first nucleic acid strand is immobilized at an end thereof on a solid-phase surface.

9. The method according to claim 7, wherein the dissociation of said complementary strand into said single strands is detected by measuring a change in a dielectric constant available from a dielectric held on said second nucleic acid strand.

10. The method according to claim 7, wherein the dissociation of said complementary strand into said single strands is detected by measuring a change in a weight of said detecting nucleic acid strand.

11. The method according to claim 7, wherein the dissociation of said complementary strand into said single strands is detected by measuring a reduction in an intensity of fluorescence from an intercalator bound or adsorbed on said complementary strand of said first and second base sequence regions in said detecting nucleic acid strand and capable of emitting fluorescence.
